(19) United States
(12) Patent Application Publication
(10) Pub. No.: US 2008/0213867 A1
(43) Pub. Date: Sep. 4, 2008

(54) Liquid Media for Chlamydospore Production of the Fungus Pochonia Chlamydsporia

(75) Inventor: Carlos Manuel Atunes Franco, Evora (PT)

Correspondence Address: SUGHRUE MION, PLLC, 2100 PENNSYLVANIA AVENUE, N.W., SUITE 800, WASHINGTON, DC, 20037, US

(73) Assignee: UNIVERSIDADE DE EVOA, Evora (PT)

(21) Appl. No.: 12/064,152

(22) PCT Filed: Sep. 13, 2006
(86) PCT No.: PCT/IB06/53257

§ 371 Date: Feb. 19, 2008

(30) Foreign Application Priority Data
Sep. 13, 2005 (PT) 103346

(51) Int. Cl. C12N 1/00

(52) U.S. Cl. 435/256.8

(57) ABSTRACT

This invention refers to the creation of liquid media to grow the fungus Pechonica chlamydosporia designed to produce conidia of the aleuriospore type, also denominated chlamydospores. These media using easily carried out fermentation processes, achieved productions over 10⁶ chlamydospores/ml. The chlamydospores can then be easily extracted using processes of filtration and separation of solid particles in liquid suspensions. These chlamydospores can be mass collected and utilized to formulate products to use in biological control of Meloidogyne spp. by the nemathophagous fungus P. chlranaydosporia, either in small scale trials or at commercial scale.

### TECHNICAL FIELD OF THE INVENTION

This invention relates to the production of chlamydospores of the fungus Pochonia chlamydosporia in liquid media. Relates also with the nutrients and composition of these liquid media. These media can be completely synthetic and so constituted by successive addition of single pure nutrients, or they might be complex mixtures obtained through extraction from biological products which have the ability to produce chlamydospores of the fungus Pochonia chlamydosporia. This invention relates also with the composition of liquid media to produce more then 10⁴chlamydospores/ml of media.

### BACKGROUND ART

At present, the production of chlamydospores with the fungus Pochonia chlamydosporia its only possible using solid media, as explained in detail in the IOBC/OILB research manual (Kerry & Bourne, 2002). For laboratory purposes, its possible to produce chlamydospores using agar media, being CMA (Corn Meal Agar) the medium most commonly used (Kerry & Bourne, 2002).

The mass production of chlamydospores is normally based on solid media obtained from cereal grains, typically barley, corn (Kerry & Bourne, 2002) or rice (Atkins et al., 2003). It can be used whole grains, broken grains or ground grains, alone or mixed with inert material in order to improve the physical properties of the medium.

The use of liquid media to produce chlamydospores is either not possible or the production is very small (less than 10³chlamydospores/ml of medium) depending on the media used. There are no published patents concerning the use of liquid media in the production of chlamydospores with the fungus Pochonia chlamydosporia. At present, there is no known liquid medium capable of producing large amounts (greater than 10³chlamydospores /ml of medium) of chlamydospores of the fungus Pochonia chlamydosporia (Liu, X. Z. & Chen, S. Y., 2003; Mo, M. et al., 2005).

### Solutions Provided by This Invention

The main problem solved with this invention has to do with the impossibility, before that, to produce chlamydospores in liquid media. This invention is characterized in that supplies the composition of liquid media to grow the fungus Pochonia chlamydosporia for the production of chlamydospores in submerged liquid culture, obtained by mixtures of pure substances or from mixtures of compound products of animal or vegetable origin, which contain globular proteins, lipids and mineral salts in its composition.

Related to the problem mentioned above, there was the impossibility, before this invention and using liquid media systems for mass production of Pochonia chlamydosporia, to produce chlamydospores in large amounts (above 10³ chlamydospores/ml of medium). This invention is characterized in that it supplies the composition of liquid media suitable for the production of large amounts of chlamydospores and by supplying the composition of media suitable for productions greater than 10⁶chlamydospores/ml of medium.

### DETAILED DESCRIPTION

The liquid medium developed is for use as substrate for the submerged micelial growth and following submerged production of chlamydospores (dictyoclamidospores or conidia of aleuriospore type) of the fungus Pochonia chlamydosporia. The components of the medium are mixed with distilled water, being, most of it, solids although some of them might have a liquid or gaseous nature. These components make water solutions, emulsions or suspensions depending on the type of substances utilized, and the final mixture is a complex emulsion where the amphiphilic and polar substances enfold those components of smaller polarity. Nevertheless, the media made this way produce stable mixtures, generally with low turbidity at room temperature and pressure (10-25° C. and approximately 1 atm). All the media have to be sterilised before being inoculated with Pochonia chlamydosporia. This is important because these media are rich in nutrients and thus support the growth of an enormous variety of organisms namely those contaminating the media constituents.

Types of substances which are part of the final composition of the medium:
Globular proteins, phospholipids, triglycerides, soluble sugars and mineral salts.

Description of the substances that enter the composition of the medium and its adequate concentration:
Globular proteins: albumin and/or casein and/or other globular proteins—between 0.05 g/l and 500 g/l;
Phospholipids: Phosphatidylcholine and/or Phosphatidylethanolamine and/or other complex mixtures of phospholipids such as lecithin—between 0.05 g/l and 100 g/l;
Triglycerides: sunflower oil or any other vegetable oil—0 g/l and 100 g/l;
Soluble sugars: saccharose and/or other disaccharides and/or glucose and/or other monosaccharide and/or other soluble sugars—0 e 50 g/l;
Mineral salts:
a) Calcium between 0 and 25 mM;
b) Chlorine between 0 and 25 mM;
c) Sodium between 0 and 50 mM;
d) Magnesium between 0 and 25 mM;
e) Sulphate between 0 and 25 mM;
f) Potassium between 0 and 50 mM;
g) Phosphate between 0 and 25 mM;
h) Iron between 0 and 5 mM; h) Nitrate between 0 and 50 mM.

Example of a liquid medium that yields a chlamydospore production greater then 105 chlamydospores/ml of medium with the following composition:
Albumin or Casein—1 g/l
Lecithin or Phosphatidylcholine—1 g/l
Sunflower oil—1 g/l
Sodium chloride (NaCl)—0.4 g/l
Magnesium sulphate heptahydrate (MgSO₄.7H₂O)—0.5 g/l
Dipotassium phosphate (KH₂PO₄)—1 g/l
Sodium hydroxide (NaOH)—0.2 g/l
Potassium sulphate (K₂SO4)—0.6 g/l
Iron sulfate (FeSO₄.7H₂O)—0.02 g/l

The media may have complex mixtures, such as products of vegetable origin, or of animal origin, as long as the components referred to above enter their composition according the minimum quantities specified.

Examples of products of vegetable origin: extracts or suspensions from cereals, protein rich grains, oil rich grains, or other plants such as corn, wheat, oats, barley, rice, bean, pea, broad bean and soybean.

Examples of products of animal origin: products produced from eggs and milk of any animal, their by-products, or other product mixtures from animal origin of any kind.

### Examples of Application

Development and production in laboratory of chlamydospores strain PcMR (see International Patent request no. WO2005121314) of the fungus Pochonia chlamydosporia.

A—Use of the Medium Named AL (Albumin+Lecithin Medium)

TABLE 1
Composition of salt mixture type A
Composition of the mixture of salts type A

| Mixture of salts type A | Composition (g/l) |
|---|---|
| NaNO₃ | 1.15 |
| KNO₃ | 0.70 |
| Mg(NO₃)₂•6H₂O | 0.40 |
| Ca(OH)₂ | 0.60 |
| NaOH | 0.10 |
| MgSO₄•7H₂O | 0.50 |
| FeSO₄•7H₂O | 0.02 |
| K₂SO₄ | 0.40 |
| H₃PO₄ | 0.65 |
| KH₂PO₄ | 0.30 |
| KCl | 0.20 |
| NaCl | 0.30 |
| Total | 5.32 |

Composition of AL medium utilized (g/L):
3.25 g of Albumin (of chicken egg, type II, Sigma-Aldrich)+1 g of soya lecithin +5.32 g of salt mixture type A (Table 1).

Methods and Results Obtained with AL Medium

The yields were obtained in a culture of 5 Erlenmeyer flasks of 250 ml, each with 100 ml of AL medium, inoculated with 1 ml of a 10 days old PcMR culture grown at 25° C., in Czapek medium (Table 2) where no chlamydospores are produced.

TABLE 2
Composition of Czapek medium

| Czapek | Composition (g/L) |
|---|---|
| Saccharose | 30.00 |
| NaNO₃ | 3.00 |
| MgSO₄•7H₂O | 0.50 |
| KCl | 0.50 |
| KH₂PO₄ | 1.00 |
| FeSO₄•7H₂O | 0.02 |
| Yeast extract | 0.50 |
| Total | 35.52 |

The static culture was kept for 30 days at 25° C., shaken once a day. The results (Table 3) allow the utilization of the AL medium to produce chlamydospores in laboratory for laboratory purposes.

TABLE 3
Results obtained with AL medium.

| AL medium | Sample | Counting (chlamydospores/ml) |
|---|---|---|
| Flask 1 | 1 | 1.7E+05 |
| | | 1.4E+05 |
| | 2 | 9.0E+04 |
| | | 1.2E+05 |
| | 3 | 8.0E+04 |
| | | 1.1E+05 |
| Flask 2 | 1 | 6.0E+04 |
| | | 1.1E+05 |
| | 2 | 7.0E+04 |
| | | 6.0E+04 |
| | 3 | 8.0E+04 |
| | | 5.0E+04 |
| Flask 3 | 1 | 1.4E+05 |
| | | 1.8E+05 |
| | 2 | 1.1E+05 |
| | | 7.0E+04 |
| | 3 | 1.3E+05 |
| | | 9.0E+04 |
| Flask 4 | 1 | 6.0E+04 |
| | | 1.3E+05 |
| | 2 | 1.4E+05 |
| | | 1.8E+05 |
| | 3 | 7.0E+04 |
| | | 8.0E+04 |
| Flask 5 | 1 | 1.2E+05 |
| | | 1.1E+05 |
| | 2 | 1.1E+05 |
| | | 1.3E+05 |
| | 3 | 9.0E+04 |
| | | 7.0E+04 |
| Mean | — | 1.1E+05 |

B—Use of the Medium Named OS (Egg+Sugar Medium)

Composition of the OS medium utilized (g/L):
106 grams of chicken egg (white +yolk)+30 g of saccharose.

Procedures and Results Obtained With the OS Medium

The results were obtained in a culture of 5 Erlenmeyer flasks of 250 ml, each with 100 ml of OS medium, inoculated with 1 ml of a 10 days old PcMR culture grown at 25° C., in a Czapek medium (Table 3) where no chlamydospores are produced. The static culture was kept for 30 days at 25° C., shaken once a day.

The results (Table 4) allow the utilization of the OS medium to produce chlamydospores for laboratory or industrial purposes.

TABLE 4
Results obtained with OS medium.

| OS medium | Sample | Counting (chlamydospores/ml) |
|---|---|---|
| Flask 1 | 1 | 1.1E+06 |
| | | 8.4E+05 |
| | 2 | 1.0E+06 |
| | | 1.2E+06 |
| | 3 | 1.2E+06 |
| | | 8.8E+05 |
| Flask 2 | 1 | 1.4E+06 |
| | | 1.6E+06 |
| | 2 | 2.7E+06 |
| | | 1.7E+06 |
| | 3 | 2.4E+06 |
| | | 2.1E+06 |
| Flask 3 | 1 | 2.3E+06 |
| | | 2.4E+06 |
| | 2 | 3.7E+06 |
| | | 2.8E+06 |
| | 3 | 2.4E+06 |
| | | 1.9E+06 |
| Flask 4 | 1 | 1.9E+06 |
| | | 1.1E+06 |
| | 2 | 1.2E+06 |
| | | 2.0E+06 |
| | 3 | 1.6E+06 |
| | | 1.3E+06 |
| Flask 5 | 1 | 1.6E+06 |
| | | 8.8E+05 |
| | 2 | 2.0E+06 |
| | | 2.0E+06 |
| | 3 | 1.4E+06 |
| | | 1.0E+06 |
| Mean | — | 1.7E+06 |

### INDUSTRIAL APPLICATION

These media can be used for the mass production of chlamydospores of the fungus Pochonia chlamydosporia, utilising submerged liquid culture in fermentors of various sizes, and using a strain of Pochonia chlamydosporia with industrial interest, namely a strain selected to be used in biological control of the root-knot-nematodes (Meloidogyne spp.). This technology for the production of chlamydosporia in large scale might be complemented with extraction methods for solid particles from liquid suspensions (see International Patent request no. WO2005115584), aiming to mass produce chlamydospores which then might be formulated for applications in biological control.

### REFERENCES

Atkins, Simon D., Hidalgo-Diaz, Leopoldo, Clark, Ian M. Morton, C. Oliver, Oca, Nivian Montes de, Gray, Paul. A. and Kerry, Brian. R. (2003); Approaches for monitoring the release of Pochonia chlamydosporia var. catenulata, a biocontrol agent of root-knot nematodes; Mycology Research; 107(2): 206-212.

Liu, Xingzhong Z. and Chen, Senyu Y. (2003); Nutritional requirements of Pochonia chlamydosporia and ARF18; fungal parasites of nematode eggs; Journal of Invertebrate Technology; 83: 10-15.

Kerry, B. R. and Bourne, J. M. (ed.) (2002) A Manual for Research on Verticillium chlamydosporium, a Potential Biological Control Agent for Root-Knot Nematodes. IOBC/WPRS, Gent, Belgium, 84 pp.

Mo, MingHe, Xu, ChuanKun and Zhang, KeQin (2005); Effects of Carbon and Nitrogen sources, carbon-to-nitrogen ratio, and initial pH on the growth of nematophagous fungus Pochonia chlamydosporia in liquid culture; Mycopathologia; 159: 381-387.

1. Liquid Media to grow in culture the fungus Pochonia chlamydosporia characterized in that they originate the production of conidia of aleuriospore type also designated dictyochlamydospores or simply chlamydospores, achieving chlamydospore yields over 10⁴ chlamydospores/ml and by possessing in its composition an amount of proteins over 0.05 g/l and an amount of lipids over 0.05 g/l.

2. Liquid Media to grow in culture the fungus Pochonia chlamydosporia according to claim 1, characterized by having a protein content over 0.05 g/l, but preferably between 1 g/l and 100 g/l.

3. Liquid Media to grow in culture the fungus Pochonia chlamydosporia according to claim, characterized by having in its composition proteins of any kind, but preferably globular proteins, extracted from any kind of organism, such as albumin, casein or others with similar structure and with a final shape approximately globular.

4. Liquid Media to grow in culture the fungus Pochonia chlamydosporia according to claim 1, characterized by having a lipid content over 0.05 g/l, but preferably between 1 g/l and 100 g/l.

5. Liquid Media to grow in culture the fungus Pochonia chlamydosporia according to claim 1, characterized by having in its composition lipids of any kind, but preferably phospholipids or triglycerides, or a mixture of both kinds, extracted from any kind of organism.

6. Liquid Media to grow in culture the fungus Pochonia chlamydosporia according to claim 1, characterized by having phospholipids preferably phosphatidylcholine, phosphatidylethanolamine or a mixture of phospholipids that includes just these or these and others.

7. Liquid Media to grow in culture the fungus Pochonia chlamydosporia according to claim 1, characterized by having triglycerides preferably rich in fatty acids such as oleic, linoleic, palmitic and stearic, although might also have other fatty acids, provided they can be added to the liquid medium.

8. Liquid Media to grow in culture the fungus Pochonia chlamydosporia according to claim 1, characterized by having in its composition mineral salts, but preferably the types and concentrations presented below:
a) Calcium between 0 and 25 mM;
b) Chlorine between 0 and 25 mM;
c) Sodium between 0 and 50 mM;
d) Magnesium between 0 and 25 mM;
e) Sulfate between 0 and 25 mM;
f) Potassium between 0 and 50 mM;
g) Phosphate between 0 and 25 mM;
h) Iron between 0 and 5 mM;
h) Nitrate between 0 and 50 mM.

9. Liquid Media to grow in culture the fungus Pochonia chlamydosporia according to claim 1, characterized by a production of chlamydospores over 10⁶ chlamydospores/ml and by having in its composition, carbohydrates, mineral salts, proteins and lipids.

10. Liquid Media to grow in culture the fungus Pochonia chlamydosporia according to claim 1, characterized by having in its composition, the following elements:
a) 1 to 50 g/l of a source of carbohydrates, such as saccharose, glucose or others;
b) 1 to 50 g/l of a source of globular proteins, preferably proteins such as albumin, casein or others;
c) 0.1 to 10 g/l of a source of phospholipids preferably phosphatidylcholine, lecithin or others;
d) 0.1 to 10 g/l of a source of triglycerides, preferably liquid at room temperature and rich in fatty acids such as oleic, linoleic, palmitic and stearic, although their content might include other fatty acids;
e) a mixture of salts, preferably containing the types and concentrations presented below:
e1) Calcium between 1 and 25 mM;
e2) Chlorine between 5 and 25 mM;
e3) Sodium between 5 and 50 mM;
e4) Magnesium between 5 and 25 mM;
e5) Sulfate between 5 and 25 mM;
e6) Potassium between 5 and 50 mM;
e7) Phosphate between 5 and 25 mM;
e8) Iron between 0.01 and 5 mM;
e9) Nitrate between 0 and 50 mM.

11. Liquid Media to grow in culture the fungus Pochonia chlamydosporia according to claim 1, characterized by having in its composition products of vegetable origin, preferably extracts or suspensions of cereal seeds, protein seeds, oil seeds or other plant seeds such as corn, wheat, oat, barley, rice, bean, pea, broad bean and Soybean.

12. Liquid Media to grow in culture the fungus Poehonia chiamydosporia according to claim 1, characterized by having in its composition products of animal origin, preferably originated from eggs or milk from any animal species and its by-products or mixtures of animal products of any origin.
